(19) United States
(12) Patent Application Publication
(10) Pub. No.: US 2009/0280559 A1
(43) Pub. Date: Nov. 12, 2009

(54) GENETIC SEQUENCER INCORPORATING FLUORESCENCE MICROSCOPY

(75) Inventor: Kevin McCarthy, Plaistow, NH (US)

Correspondence Address: VEDDER PRICE P.C., 222 N. LASALLE STREET, CHICAGO, IL, 60601, US

(73) Assignee: KOLLMORGEN CORPORATION, Northampton, MA (US)

(21) Appl. No.: 12/434,425

(22) Filed: May 1, 2009

Related U.S. Application Data
(60) Provisional application No. 61/050,759, filed on May 6, 2008.

(51) Int. Cl. C12M 1/00; G05B 19/40; G02B 27/10; G03B 13/34

(52) U.S. Cl. 435/287.2; 318/685; 359/629; 396/133

(57) ABSTRACT

A fluorescence microscopy sequencer comprises a fluid transport subsystem in which reagents are pumped through a series of multi-port valves to a mixer or one or more flow cells, or directly into the flow cell(s). The one or more multi-port valves can be mounted upon a fluids manifold having syringe tubes mounted on the opposite side. Mounted on a movable support, the manifold may be brought into and out of fluid communication with a storage block comprising the plurality of reagents. In another embodiment, the sequencer comprises a beamsplitter indexer that facilitates the quick and reliable switching of filter cubes through use of a stepper motor. In yet another embodiment, a motion control system is provided in which an inertial reference is interposed between and directly coupled to a first and second axis of control, thereby minimizing any low structural resonant frequencies and enabling high performance (high frequency response) motion control.

This application claims priority to the provisional patent application having Application No. 61/050,759, filed May 6, 2008, having inventor Kevin McCarthy, entitled GENETIC SEQUENCER INCORPORATION FLUORESCENCE MICROSCOPY.

### FIELD OF THE INVENTION

The instant disclosure relates generally to equipment used in the study of molecular biology, genomics, bioinformatics and the like and, in particular, to a sequencer incorporating fluorescence microscopy.

### BACKGROUND OF THE INVENTION

Over the last thirty years, remarkable advances have been made in decoding the genomes (the encoded form of all hereditary traits) for a variety of organisms, from simple viruses to human beings and other mammals. Various tools have been developed over the years to assist with this decoding or sequencing of genomes. Given the sheer complexity of genomes for more complex organisms (such as humans), the time and cost involved in sequencing such genomes has been quite high. For example, the well-known Human Genome Project required 13 years and $3.5 B to sequence the first human genome in 2003, and would incrementally cost approximately $300M to repeat today. However, advances in sequencing techniques and equipment have led to corresponding improvements in the speed and cost in performing such sequencing. For example, in September 2007, the second human genome was published, having taken one year and $70M to complete. Concurrently, so-called second generation sequencing techniques (based on the analysis of shorter, random segments of DNA (deoxyribonucleic acid) strands and subsequent reassembly based on computationally-intensive comparisons of overlapping portions of the different sequences) have been developed that offer the potential to improve both the speed and expense of sequencing.

Many sequencing techniques rely on fluorescence microscopy in which the properties of organic or inorganic substances are studied using the phenomena of fluorescence. A component of interest in a specimen is specifically “labeled” with a fluorescent molecule called a fluorophore and illuminated with light of a specific wavelength causing the fluorophore to emit longer wavelengths of light (i.e., of a different color than the absorbed light). The illumination light is separated from the much weaker emitted fluorescence through the use of appropriate filters and an image taken of the emitted light. By studying such images, it is possible to identify and determine the properties of the specific substances (e.g., DNA nucleotides).

Regardless of the specific techniques used to perform sequencing, a continuing impediment to more widely available genetic sequencing (and it's potential benefits such as personalized, genetics-based medical care) is the prohibitive cost of the equipment and consumables used to perform sequencing. For example, current second generation sequencing machines typically cost in the range of approximately $450K to $1.35M. Furthermore, while somewhat less expensive, prior generation sequencing machines do not operate as efficiently as second generation machines and offer dramatically reduced throughput in comparison. In short, while rapid and significant advances have been made in the molecular biology and organic chemistry needed to accurately and efficiently perform genomic sequencing, the development of suitable sequencing platforms has failed to keep pace.

### SUMMARY OF THE INVENTION

The instant disclosure describes a fluorescence microscopy-based genomic sequencer that realizes a dramatic reduction in initial equipment cost without, it is believed, any loss in performance. In this manner, the benefits of widespread genetic sequencing may be more readily realized. Generally, the disclosed sequencer implements a fluorescence microscope system in which reagents flow through a fluids subsystem to a mixer prior to testing, unlike prior art devices in which relatively expensive and bulky autosamplers are used to bring reagents together and place them in a testing location. As used herein, reagents is a generic term that includes both specialized organic compounds, washes, simple inorganic solutions, and solvents, including water, used in operation of the disclosed sequencer. In one embodiment, the disclosed system for transporting fluids comprises a multi-port pump in fluid communication with one or more multi-port valves that, in turn, are in fluid communication with storage for a plurality of reagents. Under suitable processor-based control, the multi-port pump causes reagents to be drawn through the one or more multi-port valves into either the mixer and then one or more flow cells, or directly into the flow cell(s). Similarly, the multi-port pump can draw fluids from the flow cell(s) to a waste container. In an embodiment, the one or more multi-port valves are mounted upon one side of a fluids manifold having syringe tubes mounted on the opposite side. Mounted on a movable support, the manifold may be brought into and out of fluid communication with a storage block comprising the plurality of reagents, thereby facilitating quick and efficient servicing of the sequencer.

In another embodiment, the sequencer comprises a beamsplitter indexer that facilitates the quick and reliable switching of so-called filter cubes. In particular, a support member for a plurality of filter cubes is provided and coupled, directly or otherwise, to a suitable stepper motor. The support member further comprises an index indicator that cooperates with a sensor to determine an initial position of the support member, thereby ensuring consistent and reliable switching of filter cubes.

In yet another embodiment, a motion control system is provided in which a first and second axis of control, for controlling motion of an objective and target platform relative to one another along respective, perpendicular axes, are coupled directly to an inertial reference. A third axis of control, for controlling motion of the target platform along a third axis perpendicular to both the first and second axes, is coupled to the second axis of control. Because the first and second axes of control are coupled directly to the inertial reference, high performance (high frequency response) motion control can be achieved more readily while minimizing the deleterious low-frequency resonance effect of any structural connections between the axes of control and the inertial reference.

### BRIEF DESCRIPTION OF THE DRAWINGS

The features described in this disclosure are set forth with particularity in the appended claims. These features and attendant advantages will become apparent from consideration of the following detailed description, taken in conjunction with the accompanying drawings. One or more embodiments are now described, by way of example only, with reference to the accompanying drawings wherein like reference numerals represent like elements and in which:

FIG. 1 is a schematic block diagram of a fluorescence microscopy sequencer in accordance with the instant disclosure;

FIG. 2 is a perspective view of an internal arrangement of components of a fluorescence microscopy sequencer in accordance with the instant disclosure;

FIG. 3 is a schematic illustration of a system for transporting fluids in accordance with the instant disclosure;

FIG. 4A is a perspective view of an implementation of a portion of the system for transporting fluids illustrated in FIG. 3 and in which the system is illustrated in an “open” position;

FIG. 4B is an enlarged, perspective view of an embodiment of a storage block illustrated in FIG. 4A and used for the storage of reagents;

FIG. 4C is a top view of the storage block illustrated in FIG. 4B;

FIG. 5 is a perspective view of the implementation of the portion of the system for transporting fluids illustrated in FIG. 4A and in which the system is illustrated in a “closed” position;

FIG. 6 is a perspective view of pump and mixer arrangement in accordance with the instant disclosure;

FIG. 7 is a partial cutaway and perspective view of the mixer illustrated in FIG. 6;

FIG. 8 is a perspective view of a beamsplitter indexer in accordance with the instant disclosure;

FIGS. 9 and 10 illustrate various potential arrangements for components of a motion control system relative to an inertial reference;

FIG. 11 illustrates a presently preferred arrangement for components of a motion control system relative to an inertial reference;

FIG. 12 is a perspective view of an implementation of the arrangement illustrated in FIG. 11; and

FIG. 13 is a side view of the implementation illustrated in FIG. 12.

### DETAILED DESCRIPTION OF THE PRESENT EMBODIMENTS

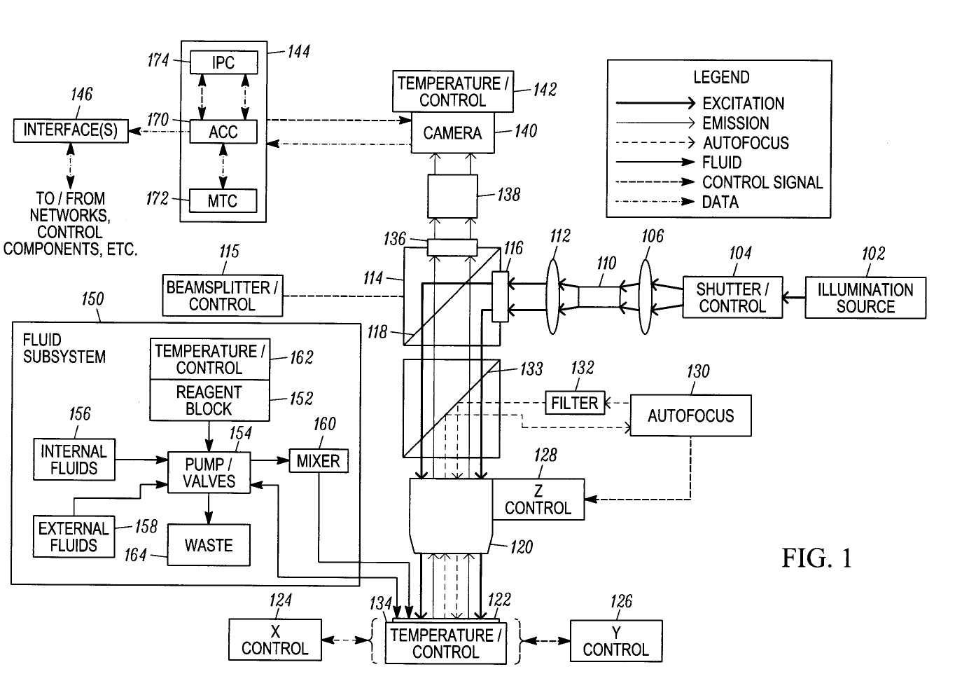

Referring now to FIG. 1, a schematic block diagram of a fluorescence microscopy sequencer 100 is illustrated in simplified form. An illumination source 102 is provided as the source for excitation illumination to be provided to samples for analysis. In a presently preferred embodiment, the illumination source 102 comprises a 300-watt, forced air-cooled xenon lamp. However, the instant disclosure is not limited to any particular illumination source, which may be selected as a matter of design choice. A xenon lamp offers the benefit of a relatively flat operating spectrum as compared to other illumination sources, thereby allowing the selection of excitation illumination (to accommodate different fluorophores) to be simplified to the appropriate selection of an excitation filter 116. Alternatively, the xenon lamp can be replaced by one or more lasers of having suitable wavelengths tuned or tunable to the desired fluorophore absorption wavelengths. In this embodiment, four lasers of different wavelengths (e.g., one for each of the four different DNA nucleotides) are preferably provided.

A programmable shutter assembly 104 is coupled to the illumination source 102. In one embodiment, the shutter 104 includes a light entry tube to prevent stray light from entering the enclosure, a cold mirror, an infrared (IR) beam dump, a focusing lens, and a high-speed shutter vane. As known in the art, the cold mirror employs multi-layer dielectric coatings to provide very high (>95%) reflection at the wavelength range of interest (360 to 730 nm) while transmitting unwanted IR energy into the beam dump. The high-speed, low-inertia shutter vane intercepts the converging cone of light just before entrance to a liquid light guide 110 aperture. The travel, acceleration, deceleration, and velocity of the shutter can be flexibly programmed via a motion controller 172 (described below). Operating in conjunction with the camera 140 (described in greater detail below), the shutter's primary purpose is not to set the exposure duration, but rather to rapidly open and then extinguish light on either side of the camera's integration period, ensuring that virtually all photons hitting the sample are usefully integrated by the detector, and do not contribute to photobleaching of the sample outside of the integration period.

A focusing lens 106, preferably made from BK-7 optical glass, is anti-reflection coated to maximize light transmission, and focuses the collimated beam from the shutter 104 into the aperture of a liquid light guide 110, available from Lumatec GmbH, which provides an easily routed path for communicating the excitation illumination to the excitation filter 116. The internal fluid of the liquid light guide 110 is chosen to maximize its life span and transmission across the desired spectrum of wavelengths, e.g., 360 nm to 730 nm. The liquid light guide 110 also spatially homogenizes the transmitted light, permitting programmable positioning of the shutter 104 with many (up to 200) levels of intensity between fully closed and fully open, while maintaining uniform field illumination, which in turn provides an additional degree of control over sample exposure. The light guide 110 terminates in an anti-reflection coated collimating lens 112, also preferably made from BK-7 optical glass, which generates a parallel beam of light that then enters a filter cube 114.

As those having ordinary skill in the art will appreciate, high-throughput fluorescence microscopy is a dynamically choreographed, wavelength-selective parsing of light. The wavelength-selective parsing of the light, in turn, is achieved through the use of one or more optical filters, often referred to as filter cubes 114. In an embodiment, a plurality of filter cubes 114 are provided. As described in greater detail below, filter cubes 114 may be provided as sets of four cubes. A beamsplitter indexer and control 115 quickly positions any of the four wavelength selective filter cubes 114, or a companion (fifth) white light filter cube used for positioning of samples prior to analysis, into precise position along the optical axis.

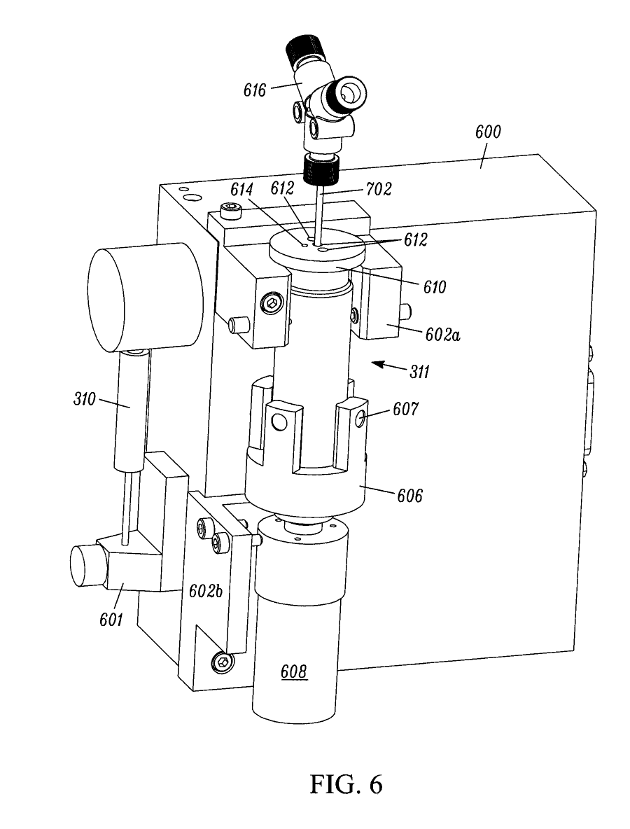

Each wavelength-selective filter cubes 114 typically comprise injection molded plastic (see, e.g., FIG. 8) comprising an excitation filter 116, which narrows the light provided by the illumination source 102 to only that wavelength range needed to excite the intended fluorophores. A 45 degree dichroic beamsplitter 118 reflects the resulting excitation wavelengths toward an objective 120 and the sample while passing any resulting emission wavelengths (from the objective 120) through to an emission filter 136. The emission filter 136 ensures that, to the maximum possible extent, only light from the emitting fluorophore is passed to the camera 140. As known in the art, the filters 116, 136 and beamsplitter 118, which may be obtained from Semrock, Inc., must be coordinated with the desired fluorophores to avoid the excitation of filter n from stimulating emission from fluorophores n−1 or n+1, as seen by the camera 140 through the emission filter 116.

The objective 120 sets the desired level of magnification, resolution and light collection efficiency in conjunction with the tube lens 138. While suitable optical elements of this type for use in this application will be readily apparent to those of ordinary skill in the art, in a presently preferred embodiment, the objective 120 comprises a high performance, infinity corrected, brightfield 20×, 0.70 (or higher) numerical aperture objective coupled to a mating 1.25× tube lens, both manufactured by Leica Microsystems GmbH, for a total magnification of 25×. Use of a very high numerical aperture for the objective 120 ensures highly efficient signal collection from faint fluorophores. However, it is understood that any of a number of infinity corrected objectives may be used to accommodate specific application-dependent requirements. The optical path between the objective 120 and the camera 140 is protected against stray light, with optical black flock paper lining the objective and camera tubes.

The camera 140 is highly sensitive to the faint quantities of light emitted by the fluorophores and is preferably a digital camera that employs electron multiplying, charge-coupled device (EMCCD) technology, such as the C9100-02 model manufactured by Hamamatsu Corporation. In a presently preferred embodiment, the camera 140 comprises an 8 mm×8 mm detector (a 1,000×1,000 array of 8 um square pixels) that, combined with the magnification provided by the optical elements 120, 138, provides a field of view 320 um square, with each pixel corresponding to a nominal 320 nm square region in the specimen plane. In actuality, the diffraction-limited resolution is a function of the numerical aperture of the objective 120 and the wavelength of interest; at the shortest wavelength (360 nm), the resolution is 260 nm, a close match to the geometric resolution. At the longest wavelength (730 nm), the resolution is coarser, at 520 nm. In a presently preferred embodiment, introduction of thermally-induced dark currents (i.e., noise in the acquired images) is minimized by a hermetically sealed, high vacuum detector chamber, which is continuously maintained at −50° C. by a forced air Peltier-type thermoelectric cooler 142 operating under control of the camera 140. Using an EMCCD camera 140, an internal gain from unity up to 2,000× may be applied to each image frame, which may be acquired at rates up to 30 frames per second. The frame rate and integration period (i.e., the length of time over which the emitted light is collected) are fully programmable and controlled, in a presently preferred embodiment, by a motion and temperature controller 174, which may comprise one or more suitably programmed rack-mounted computers.

As noted above, the various components of the sequencer 100 receive control signals from and/or provide data to a centralized controller 144 that, as shown, comprises a plurality of appropriately programmed, rack-mounted computers or other processing devices, with any necessary control signals routed to the appropriate components via suitable communication paths (not all shown for ease of illustration). In the illustrated embodiment, the centralized controller 144 comprises an acquisition and control computer (ACC) 170 that serves to control overall operation of the sequencer 100. The ACC 170, in turn, is in communication with an controls operation of a motion and temperature controller (MTC) 172 that serves to control operation of all hardware components, e.g., moving components, sensors, temperature control devices, etc. The ACC 170 is also in communication with an image processing computer (IPC) 174, using suitable software-implemented image processing algorithms, provides real-time image processing and quality metrics, as well as final base calls (i.e., determination of the detected DNA sequence). In a presently preferred embodiment, the IPC 174 also communicates with one or more interfaces 146 that allow the sequencer 100 to communicate with external devices, networks, etc.

For example, the ACC 170 may be a 1 U rack-mounted computer comprising a 2.4 GHz dual core Intel Core 2 Duo central processing unit (CPU) with 4 MB of L2 cache, 2 GB of double data rate 2 random access memory (DDRII RAM), a dual gigabit Ethernet port, a serial port, a Camera Link capture card in a PCI-e slot for image acquisition, and a 1 terabyte, 7200 rpm serial advanced technology attachment (SATA II) hard drive. In addition to performing all instrument control functions, the ACC 170 additionally communicates with the camera 140 to perform image capture, temporary image storage and image transmission to the IPC 174. In turn, the IPC 174 may be a similar 1 U rack mounted computer comprising a 2.4 GHz dual core Intel Core 2 Duo CPU with 4 MB of L2 cache, 8 GB of DDRII RAM, a dual gigabit Ethernet port, and two, 1 terabyte, 7200 rpm SATA II hard drives. The MTC 172, which as noted above controls all motion-related components, temperature regulation components, fluid transport components and autofocus components, as described below, preferably comprises a 3 U rack mounted computer that communicates with the ACC 170 via any combination of serial, USB and/or Ethernet ports. Techniques for programming such computers to perform the operations described herein are well known to those having ordinary skill in the art. For example, in a presently preferred embodiment, both the ACC 170 and IPC 174 may run the “LINUX” operating system and publicly available software from the Church Laboratory at Harvard Medical School.

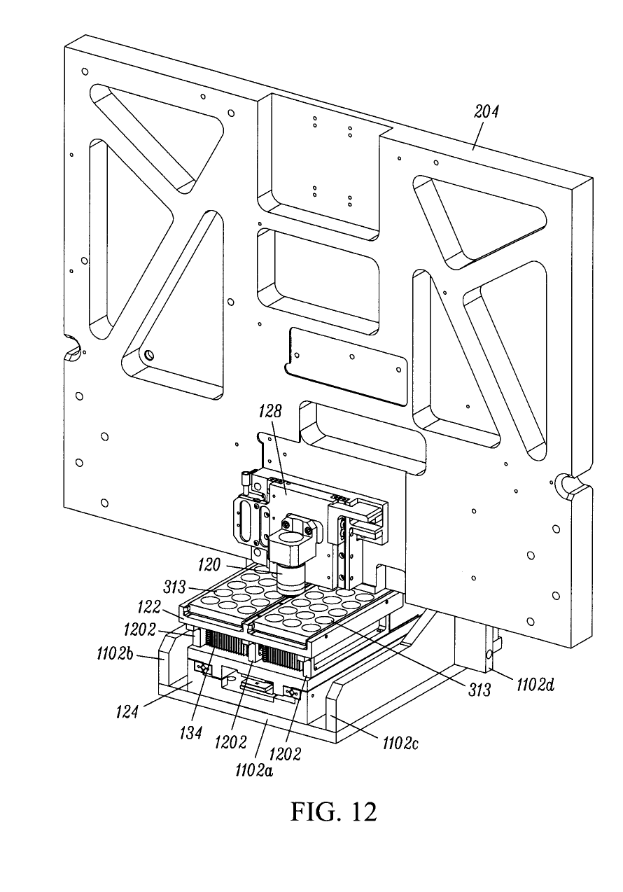

In addition to the shutter control 104 and beamsplitter indexer 115, the sequencer 100 additionally comprises so-called X-, Y- and Z-axes of control 124-128 used to precisely control positioning of a target platform 122 (comprising flow cells 313) and the objective 120 relative to one another. As shown, the X-axis 124 and Y-axis 126 preferably control motion of the target platform 122 (and corresponding temperature controller(s) 134), whereas the Z-axis 128 controls motion of the objective 120. In one embodiment, the X-, Y- and Z-axes of control 124-128 provide 150 mm×150 mm×2 mm of travel, respectively, with a resolution of 5 nm along any of the axes. Preferably, each of the axes of control 124-128 comprises a non-contact, direct-drive linear motor incorporating non-contact optical linear encoders and precision-ground crossed rollers guideways with anti-creep protection. Suitable axes of control 124-128 may be obtained from Danaher Motion—Dover. Under the control of the centralized controller, these axes provide high-performance motion, with field-to-field (a field being that portion of the sample currently being imaged) step and settle times in the X-axis closely approximating the maximum frame rate of the camera 140. As shown in greater detail with reference, for example, to FIG. 12, the target platform 122 includes a dual sample carrier, providing precision registration of sample chambers or flow cells. Additionally, temperature of flow cells 313 on the target platform 122 can be controlled by a corresponding temperature controller 134 (only one shown). In a currently preferred embodiment, each temperature controller 134 comprises a Peltier thermoelectric module operating in conjunction with active forced-air heat transfer, allowing the use of multiple temperature set points in the range of 15° C. to 60° C., with each set point being programmable for any desired time interval, thereby providing for a wide range of biochemistry protocols.

While the depth of field of the sequencer's objective 120 will necessarily depend on the configuration of the objective 120 employed, those having skill in the art will appreciate that virtually any objective 120 used for this purpose will have a very precise depth of field requiring the use of constant focus correction. For example, the depth of field of the 20×, 0.70 NA, Leica objective mentioned above varies over the wavelength range of the sequencer 100 (360 nm to 730 nm), ranging from 0.52 um at the shortest wavelength, to 1.06 um at the longest. Since it is impossible to produce and align samples with this degree of planarity, an active laser autofocus system 130 is provided. In a preferred embodiment, the autofocus system 130 uses a plane-polarized 785 nm laser diode to generate a monotonic focus error signal, which in turn is integrated by the digital Z-axis controller 128 to maintain critical, sub-micron focus at all times, including while stepping or scanning. Use of a 785 nm wavelength laser diode avoids any potential emission filter 136 attenuation of the reddest fluorophores. As noted above, resolution of the Z-axis 128 is 5 nm, and its 2 mm travel permits the objective 120 to be switched from the active autofocus plane to a retracted position during sample load and unload. Illustrated schematically, multiple wavelength selective filters 132 are used to direct the autofocus laser beam to the specimen through via a suitable beamsplitter 133, while ensuring that the autofocus laser remains utterly undetectable by the camera 140, and that the full range (360 nm to 730 nm) of potential fluorescence excitation and emission remains available at high transmission.

Prior art sequencers often rely on expensive autosamplers to mechanically access containers (often external to the actual sequencing equipment) for each of the necessary reagents prior to depositing the desired mixture on the target platform. This becomes particularly cumbersome in those applications, including genomic sequencing and live cell fluorescence imaging, that require the introduction and removal of reagents from the sample area prior to imaging. In contrast, the sequencer 100 described herein relies on a highly-flexible and efficient fluid transport system 150 to accomplish the access and mixing of reagents.

To this end, the fluid transport subsystem 150 provides storage for all reagents to be used by the sequencer 100. Generally, reagents used by the sequencer 100 are divided into two basic groups: those that require or would benefit from cooling, and those that would not. In a presently preferred embodiment, the latter 156, 158 are housed in individual Nalgene bottles within or beside the sequencer 100; in a default configuration, the sequencer 100 includes two 2 liter bottles 158, and two 250 ml bottles 156 that are connected to the rest of the fluidic subsystem via standard ⅛″ FEP tubing that slips through grommets in the screw caps of each bottle. When disposed within the sequencer 100, the liquids in these bottles 156 will be maintained at the interior temperature of the sequencer 100, typically about 31° C.

Typically, there are more reagents in the former category, i.e., those whose shelf life would be improved through controlled cooling. However, it is also typically true that the necessary volumes of such reagents tend to be considerably smaller. For this latter category of reagents, a reagent or storage block 152 is provided that allows for the storage of up to 26 individual reagents, with storage volumes ranging from 5 ml to 80 ml. As shown, a temperature controller 162, essentially identical to those described above (i.e., temperature controller 134), is provided to control the temperature of the storage block 152 at approximately 6° C. Note that, while the temperature of the storage block 152 could be set higher, this would normally not be done in practice since the objective is to prolong the shelf life of the cooled reagents within the instrument.

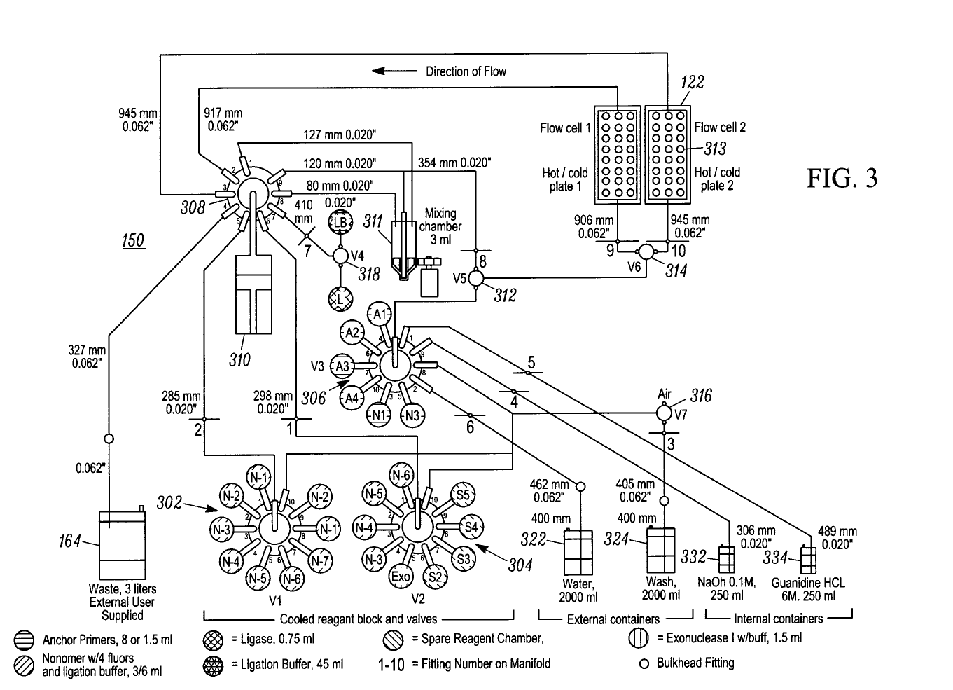

A system of pumps and valves 154, described in greater detail below relative to FIG. 3, is provided to draw the desired reagents from any of the storage components 152, 156, 158 into either a mixer 160 and then on to the flow cells 313 on the target platform, or directly to/from the flow cells 313 on the target platform 122. Similarly, the pumps and valves 154 may be used to withdraw fluids from the flow cells 313 into an externally supplied waste container 164, or from any of the storage components 152, 156, 158 into the waste container (as in the case, for example, of using water or wash to clean out any of the valves or intervening tubing). As described in greater detail below, because many of the reagents employed in genome sequencing, biochemistry experiments, etc. are very expensive, the pumps and valves 154, mixer 160 and intervening tubing is preferably kept to minimum size and lengths to minimize reagent usage.

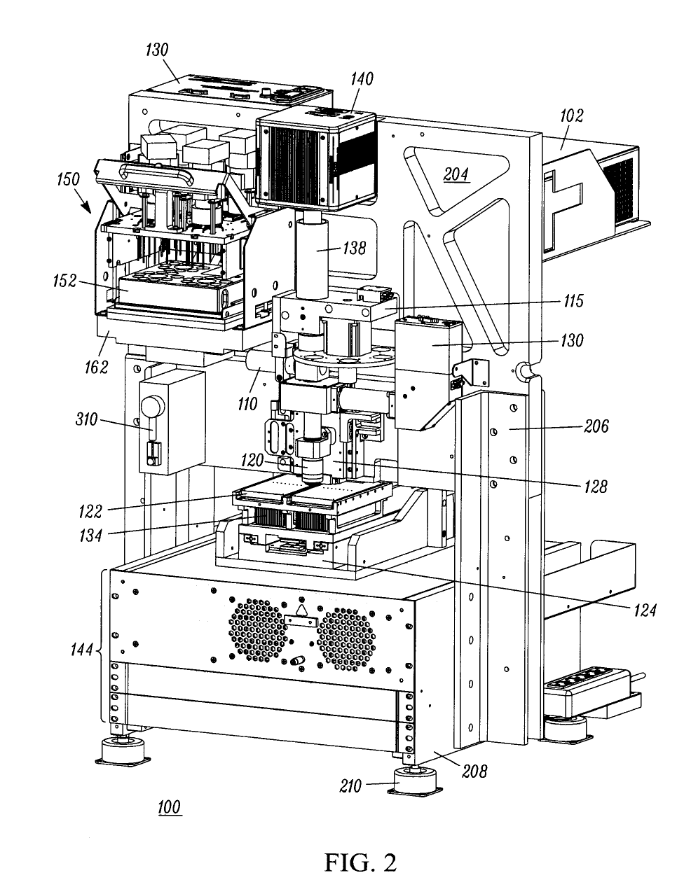

Referring now to FIG. 2, a perspective view of a presently preferred internal arrangement of various components of the fluorescence microscopy sequencer 100 is illustrated. In particular, a support frame is shown comprising an inertial reference 204 mounted to vertical supports 206 that are, in turn, mounted upon vertical walls 208 and feet 210. In a presently preferred embodiment, the inertial reference 204 is a relatively thick and heavy plate of machined aluminum that serves two primary purposes. First, the inertial reference 204 provides support to most of the components illustrated in FIG. 1; as shown, only the centralized controller 144 (preferably comprising, as noted above, the ACC 170, MTC 172 and IPC 174) is supported by the vertical walls 208. Note that the target platform 122 and temperature controller 134 sit atop the X-axis 124. The objective 120, whose vertical positioning is controlled by the Z-axis 128, sits at one end of an optical path established by beamsplitter indexer 115, tube lens 138 and camera 140. Second, the inertial reference 204 serves as a structural reference point for the various X-, Y- and Z-axes, as described in greater detail below.

Referring now to FIG. 3, an embodiment of the fluid transport subsystem 150 is illustrated in greater detail. As shown, the subsystem 150 comprises a plurality of multi-port valves 302-306 and a multi-port pump 308. The multi-port pump 308 is in fluid communication with two of the multi-port valves 302, 304, a mixer 311 and two flow cells 313. As shown, the multi-port pump 308 is capable of being configured to fluidically couple any of its ports with a syringe pump 310 and, in a similar vein, each of the multi-port valves 306-306 can be configured to fluidically couple its output port (schematically illustrated as the central port) to any of its input ports. A first three way valve 316 can be controlled to fluidically couple the third multi-port valve 306 directly to the flow cells 313, or to fluidically couple the mixer 311 to the flow cells 313. A second three way valve 314 allows either of the two flow cells 313 to be individually selected. A third three way valve 316 is fluidically coupled to all three of the multi-port valves 302-306 thereby allowing air or wash (stored in one of the external containers 158) to be drawn through any of the multi-port valves 302-306. A fourth three way valve 318 is provided in fluid communication with the multi-port pump 308 facilitating the selection of either of two specific, cooled reagents, in this case, ligase or ligation buffer (labeled L and LB, respectively). Note that suitable three-way valves are manufactured by Bürkert Fluid Control System.

As shown, the majority of the ports provided by the multi-port valves 302-306 are fluidically coupled to reagents stored in the storage block 152. In the illustrated examples, the reagents stored in this manner include anchor primers (labeled A1 through A4, N1 and N3), nonomers with 4 fluorophores and ligation buffer (labeled N−1 through N−7 and N+1 through N+6) and exonuclease (labeled Exo). In this configuration, there are four spare reagent chambers (labeled S2 through S5). As shown, both the first multi-port valve 302 and the second multi-port valve 304 are fluidically coupled exclusively to reagents stored within the storage block 152. In contrast, the third multi-port valve 306 is coupled to reagents in the storage block 152 and to either or both of the uncooled containers 156, 158 that store, in the illustrated embodiment, water, wash, sodium hydroxide or guanidine hydrochloride. Although not explicitly illustrated in FIG. 3, a manifold (described below) is used in a presently preferred embodiment to both support the multi-port valves 302-306 and the three way valves 312-318, and to establish various ones of the connections between the multi-port pump 308, the multi-port valves 302-306 and the flow cells 313.

Each of the multi-port valves 302-306 preferably comprises a ten-port rotary valve, such as those manufactured by Rheodyne LLC. The syringe pump 310, such as those manufactured by Tecan Group Ltd., is preferably equipped with a nine-port ceramic rotary valve 308 that provides volumetric flow control and additional flow routing. In a presently preferred embodiment, the syringe pump 310 volume is 1 ml with a resolution and repeatability is 0.5 ul, and an absolute accuracy of 10 ul, although it is understood that users can quickly and easily substitute a wide range of alternative syringes, trading off capacity for resolution. Given this configuration, the choice of reagents, their operational sequence, their volume, flow rate, and duration in the sample chamber at multiple temperatures, etc. all are fully programmable by a user.

With the configuration illustrated in FIG. 3, the fluid transport subsystem 150 can perform a number of operations to move fluids around within the sequencer 100. For example, the multi-port pump 308 can draw any of the reagents from the storage block 152 via either of the first or second multi-port valves 302, 304 and into the mixer 311. Note that the multi-port pump 308 provides multiple input lines (via ports 1 and 8, as illustrated) to the mixer, thereby allowing these lines from the multi-port pump 308 to the mixer 311 to be primed with frequently used reagents, if desired. As described in further detail below, the mixer 311 can be operated to mix together any fluids introduced therein. Thereafter, the multi-port pump 308 can draw the mixture from the mixer 311 into either or both of the flow cells 313 through application of a vacuum (by virtue of the syringe pump 310) to the distal end (relative to the mixer 311) of either or both of the flow cells 313. In a similar manner, those reagents available through the third multi-port valve 306 may be directly drawn into either or both of the flow cells 311, bypassing the mixer 311 altogether. To improve separation between individual reagents, the system can be configured such that wash or air may be sent through any of the multi-port valves 302-306 and the flow cells 313. Finally, the multi-port pump 308 can draw fluids from either of the flow cells 313 (or from either of the first and second multi-port valves 302, 304) to an external waste container 164.

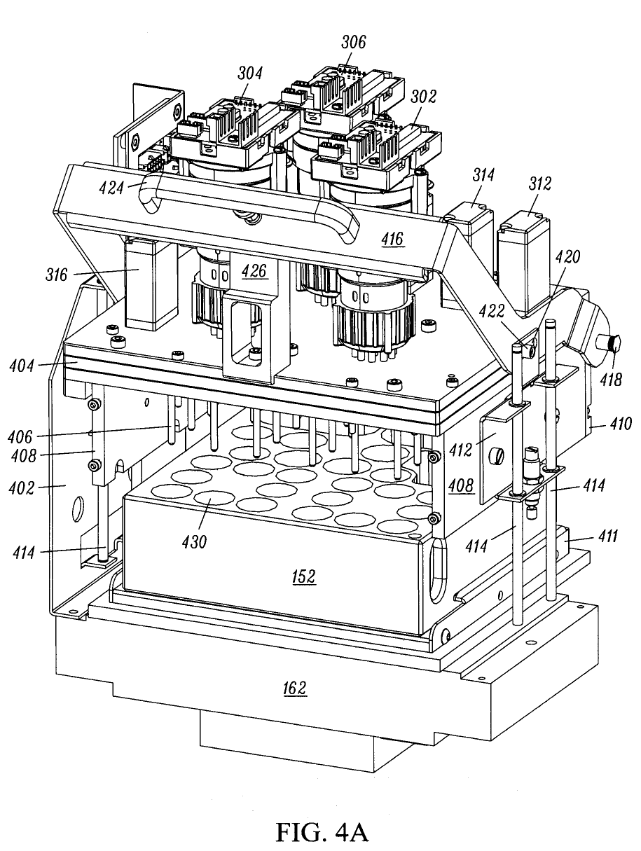

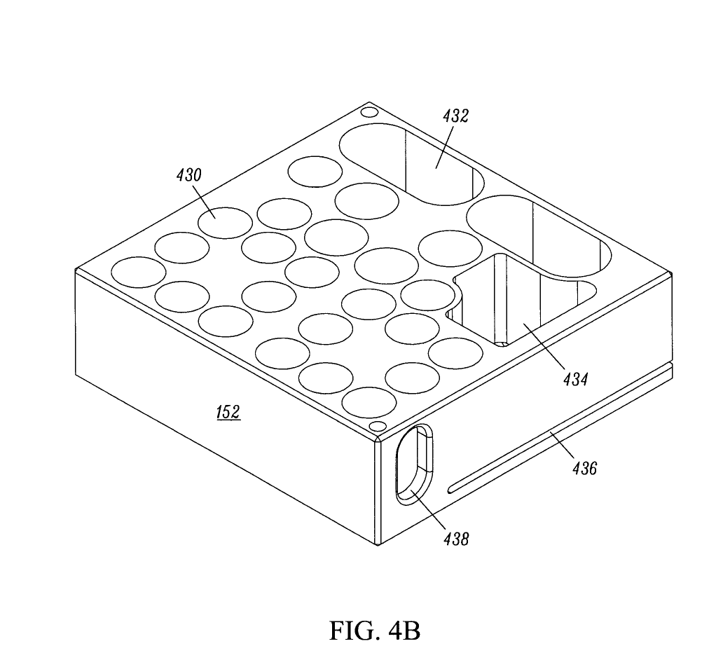

Referring now to FIGS. 4A-4C and 5, a presently preferred implementation of the fluid transport subsystem 150, including the storage block 152, is illustrated. A manifold 404 is provided, the upper surface of which serves as a platform for mounting of the multi-port valves 302-306 and three way valves 312-318. On an opposite side of the manifold 404, a plurality of stainless steel hypodermic or syringe tubes 406 extend downward and are aligned on the surface of the manifold 404 in a manner that allows them to be received by correspondingly positioned recesses or chambers 430-434 in the storage or reagent block 152. All cooled reagents are stored in a single monolithic block storage block 152, preferably made from Teflon coated aluminum (making it compatible with ultrasonic cleaning and autoclaving), that can be readily removed, cleaned, refilled, and re-inserted through a front door of an enclosure. In use, the storage block 152 sits atop the temperature controller 162 used to lower the temperature of the reagents.

As best illustrated in FIG. 4C, each recess 430-434 in the storage block 152 comprises a conically-shaped bottom surface 440. Preferably, each syringe tube 406 precisely aligns with the lowest point of each corresponding conically-shaped bottom surface 440 such that, when the manifold 404 is lowered, each syringe tube 406 is brought into fluid communication with its corresponding recess 430-434, thereby assuring that all but ˜10 ul of each reagent can be accessed. In one embodiment, each recess 430-434 can be configured to directly store a desired quantity of a corresponding reagent or, in an alternate embodiment, to store a vial or other container that in turn directly stores the desired reagent. In this latter embodiment, the internal dimensions defining each recesses 430-434 can be configured to uniquely conform to only one type of vial having a mating external surface configuration. As shown in FIGS. 4B and 4C, recesses 430-432 of varying shapes and sizes (i.e., internal volumes) may be provided as needed. For example, in a presently preferred embodiment, recesses 430-434 offering storage volumes of 0.75 ml, 1.5 ml, 3, ml, 6 ml, 8 ml and 45 ml are provided in the storage block 152. Likewise, the specific locations of each of the recesses 430-434 within the storage block 152 may be selected as necessary. In yet another embodiment, the storage block 152 may be formed of a relatively easily disposed of material, such as vacuum-formed, biologically inert plastic. Such a storage block 152 (or even an exchangeable non-disposable storage block) may be pre-filled with any desired combination of reagents and subsequently film-sealed to prevent spillage or cross-contamination of the reagents, thereby facilitating shipment of ready-to-use reagent blocks 152. In this embodiment, each syringe may be provided with knife-edge chamfers that will penetrate the sealing film when the manifold is lowered.

In the illustrated embodiment, the storage block 152 comprises lateral slots 436 that engage horizontally-disposed flanges of corresponding lateral guides 411 as the storage block 152 is inserted or removed. Hand holds 438 may also be provided in the fluid block 152 to assist with handling thereof.

In addition to providing relatively short, fluid connections between the reagents in the storage block 152 and the corresponding valves 302-306, 312-318, the manifold 404 also provides fittings that allow the connection of the uncooled containers 156, 158 to the valves 302-306, 312-318 as necessary. Furthermore, the manifold 404 may support a downwardly-projecting enclosure (only the side walls 408 and back walls 410 shown for ease of illustration) around the syringe tubes 406 that assists in providing an isothermal environment for the reagents.

As shown in FIGS. 4A and 5, the manifold 404 is preferably mounted (either directly or, as shown, via the enclosure side walls 408) on a movable support comprising a pair of brackets 412 (only one shown) that are slidably mounted on vertical guide posts 414. A lever member 416 is provided and is rotatably mounted to housing panels 402 (only one shown) via a pivot axis 418. The lever member 416 further comprises pins 422 (only one shown) that slidably engage corresponding guide slots 420 in each bracket 412. Using a handle 424 mounted on the lever member 416, the lever member 416 may be rotated about the pivots axes 418 thereby causing the pins 420 to raise or lower the brackets 412 and the attached manifold 404. FIG. 4A illustrates the manifold 404 and its movable support in the “open” position, allowing easy access to and quick insertion/removal of the storage block 152. In contrast, FIG. 5 illustrates the manifold 404 and its movable support in the “closed” position in which the syringe tubes 406 are brought into fluid communication with their corresponding recesses 430-434. Note also that, in a presently preferred embodiment, a latch post 426 is provided that engages a spring-loaded latch 428 when the manifold 404 is placed in the open position, and that must be disengaged from the latch post 426 prior to placing the manifold 404 in the closed position.

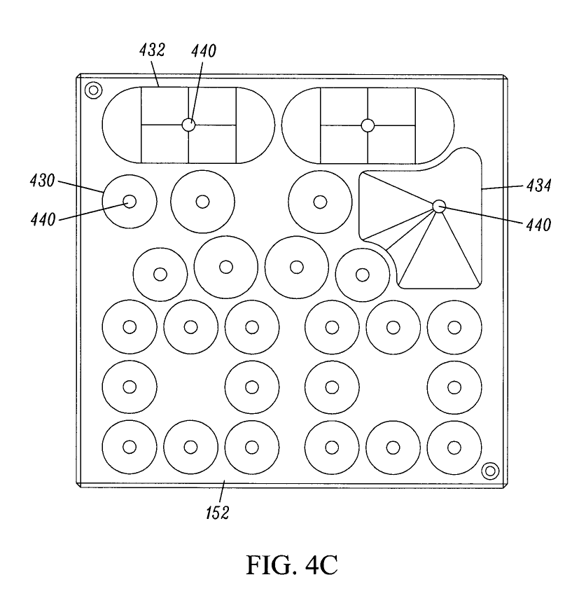

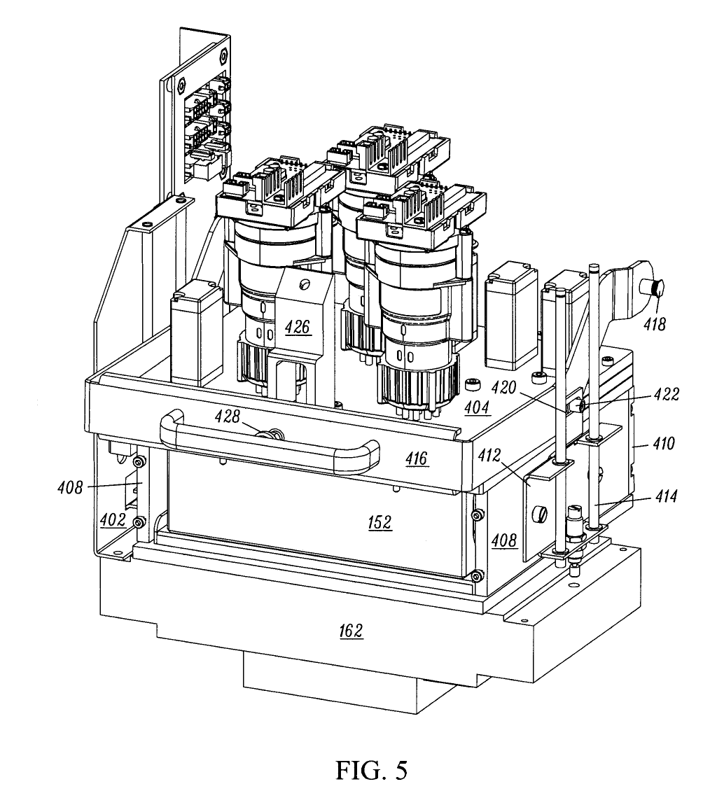

Referring now to FIGS. 6 and 7, a presently preferred embodiment of the mixer 311 is illustrated. In particular, the mixer 311 is illustrated mounted upon a pump housing 600 that includes the electronic and mechanical elements used to control the syringe pump 310. As shown, the syringe pump 310 is actuated by a vertically moving stage 601 that causes fluids to be drawn into or ejected from the syringe pump 310. The mixer 311, in turn, is mounted to the pump housing 600 by virtue of a pair of brackets 602. The mixer 311 comprises a mixing chamber 604, removably mounted in a first bracket 602a, and at least partially surrounded by a rotatable yoke 606 that is driven by a motor 608 mounted to a second bracket 602b. The yoke 606 comprises a plurality of magnets 607 that, as best illustrated in FIG. 7, act upon corresponding magnets 706 mounted in a rotating member or impeller 704. That is, as the yoke 606 is rotated by the motor 608, the mutual attraction of the magnets 607, 706 induces rotational movement in the impeller 704 as well. The motor 608 may comprise a conventional DC brush motor with gearhead or the like.

In a current implementation, the mixing chamber 604 comprises a glass vial having an internal volume of 3 ml, preferably with a conically-shaped or otherwise tapered internal bottom surface (as shown in FIG. 7) and a flat external bottom surface. A lid 610 is provided in sealing engagement with an opening in the mixing chamber 604 and is preferably formed with a plurality of ports 612 and an air vent 614. As noted above, the multiple ports 612 allow for dedicated input and/or output lines between the fluid transport subsystem 150 and the mixer 311, thereby avoiding the need to dump lines of expensive reagents when switching between reagents. In the illustrated embodiment, a single output conduit 702 is shown emerging from the mixing chamber 604 via a central port 612 and terminating in t-connector 616. Preferably, the output conduit 702 is made from a relatively inflexible material, such as stainless steel tubing or the like. As best shown in FIG. 7, the output conduit 702 is substantially aligned with a longitudinal axis of the mixing chamber and extends from a proximal end to a distal end of the mixing chamber 604 (i.e., from the opening of the mixing chamber 604 to the tip of its interior tapered bottom). Placement of one end of the output conduit 702 is proximity to the distal end of the mixing chamber 604 ensures that all but a minute amount of the mixture contained in the mixing chamber 604 can be withdrawn as needed.

As further shown in FIG. 7, the impeller 704 is coaxially aligned with the output conduit 702, which passes through a longitudinal passage of the impeller 704, thereby providing an axis about which the impeller 704 may freely rotate. Note that the distal end of the conduit 702 emerges from the bottom of longitudinal passage in the impeller 704. By virtue of both gravity and the attraction of the impeller's magnets 706 to those of the external yoke 606, the impeller 704 is preferably positioned is close proximity to the distal end of the mixing chamber 604 and, further still, each fin 711 forming the impeller 704 likewise has a tapered shape 712 to better conform to the tapered bottom of the mixing chamber 604. To enhance the mixing ability of the impeller 704, each fin 711 preferably includes openings 708 and beveled upper edges 710 to better induce turbulence in the desired mixture.

Referring now to FIG. 8, a presently preferred embodiment of the beamsplitter indexer 115 is illustrated in greater detail. Generally, the beamsplitter indexer 115 comprises a support member (in this case, a rotor) 802 upon which a plurality of beamsplitters (as illustrated, in the form of filter cubes) 114 are mounted. In the illustrated embodiment, the support member 802 includes positions for up to six filter cubes although, in practice, it may be desirable to keep at least one position open, i.e., without a mounted filter cube. Movement of the support member 802 is induced by a stepper motor 804 that, in the illustrated embodiment, induces rotational movement of the support member 802. Each of the filter cubes 114 is mounted to the support member 802 such that when the stepper motor progresses through a plurality of fixed positions, a corresponding filter cube (if present) is substantially aligned with an optical axis of a light source in one plane, in this case, the collimating lens 112 and light guide 110, and another optical axis in another substantially perpendicular plane, in this case, the tube lens 138. By virtue of the openings 805 formed in the support member 802, light received from the light source is received in a first opening of the filter cube 114 (on a lateral face of the filter cube 114) and internally reflected through a second opening (on the top face of the filter cube 114) and the adjacent opening 805 in the support member 802. Likewise, a third opening in the filter cube 114 (opposite and substantially parallel to the second opening, i.e., on the bottom face of the filter cube 114) permits emitted light (from a fluorophores) to pass through the second opening and into the tube lens 138. In an current implementation, the stepper motor 804 is capable of switching between filter cubes 114 in 150 ms with angular repeatability below 10 urad. Although a rotational embodiment is illustrated in FIG. 8, it is understood that other types of movements of support members may be equally employed. For example, rather than a rotor, the support member 802 could be embodied in a linear support member that is driven in a linear fashion.

Regardless, the support member 802 also comprise an index indicator 806 (as illustrated, in the form a notch) that cooperates with a sensor 808 (in this case, metal proximity sensor) that, in turn, determines whether the index indicator 806 is present or not. Those having ordinary skill in the art will appreciate that other types of sensors, e.g., optical sensors, may also be employed for this purpose. Note that the sensor 808 and support member 802 (by virtue of its mounting upon the stepper motor 804) are maintained in substantially fixed alignment through mutual mounting upon an alignment member 810 that also maintains the optical elements (e.g., the collimating lens 112 and tube lens 138) in alignment as well. By detecting the presence of the index indicator 806, the sensor 808 can provide an indication (e.g., an electrical signal) indicative of an initial position of the support member 802. For example, during a power up sequence or following a reset of the sequencer 100, the stepper motor 804 can be controlled to relatively slowly rotate the support member 802 until the index indicator is detected. In the illustrated embodiment, the support member 802 has a peripheral edge 803 radially spaced apart from the center (beneath the stepper motor 804) of the support member 802 and the index indicator 806 is positioned along the peripheral edge 803. However, as those having skill in the art will appreciate, the index indicator 806 may take additional forms (e.g., a detectable color or pattern, such as a bar code, etc.) that may be placed elsewhere on the support member 802 (e.g., on a top surface of the rotor) or other elements of the indexer 115 (e.g., on the filter cubes themselves). Furthermore, in the illustrated embodiment, the initial position of the support member 802 causes one of the plurality of beam splitters (if present) to be optically aligned as described above. Thus, the default position of the indexer 115 is to provide a continuous optical path. However, it may be desirable to default to a position in which a continuous optical path is inhibited, in which case, it may be desirable to place the index indicator such that none of the beamsplitters (or other optical passageways) are aligned with either optical axis when at the initial position.

As known in the art, it is necessary to control the motion of the target platform 122 being analyzed relative to the objective 120. Typically, this is achieved through the use of X-, Y- and Z-axes of control, as described above, arranged in various ways. Typically, the X- and Y-axes control lateral alignment of the target platform and the objective relative to one another, whereas the Z-axis controls vertical alignment of the target platform and the objective relative to one another. In such positioning systems, high performance (fast move and settle times, tight position stability when stopped, etc) requires high servo bandwidth (i.e., the ability to respond at high frequency to perturbations). For example, in the presently described sequencer 100, it is desirable to achieve approximately 25 images a second (with 25 corresponding movements of the target platform 122 between images and continuous tracking autofocus movements of the objective 120) with tracking performance better than the depth of field (0.5 um) during image acquisition. The limit to achieving such high performance bandwidth is usually set by the lowest resonance (natural frequency) in the system. Therefore, to get high performance/high bandwidth, the resonant frequencies in all of the motion-related components (and especially in the supporting structure, which is often large and heavy) should be relative high. To get high resonant frequencies, support structures are ideally small, lightweight, and especially stiff. Relatively long and or flexible structures connecting the various axes of control would result in very low performance.

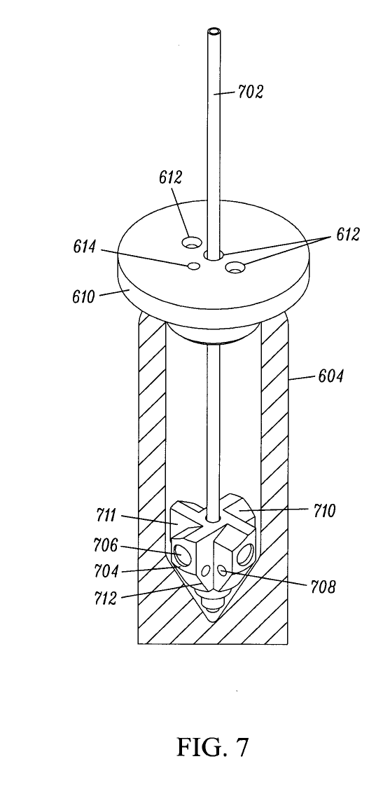

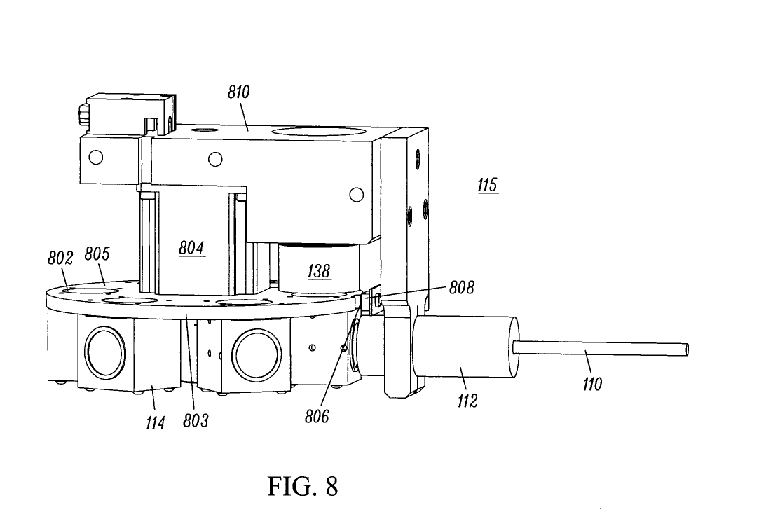

Examples of typical approaches to this are illustrated in FIGS. 9 and 10. As shown, an inertial reference 902 is provided. As used herein, an inertial reference provides a point of reference against which all controlled movements are performed. As such, an inertial reference preferably has a relatively large mass (thereby inducing a correspondingly large moment of inertia) and stiffness (thereby having a relatively high resonant frequency). Typically, the inertial reference 902 is disposed in a horizontal fashion, as shown, so as to form a surface upon which the movement components can be mounted. In a first embodiment, the various axes 904-908 are used to directly control movement of the target platform 909 relative to a substantially stationary objective 910. To this end, a first axis (in this case, the X-axis 904) is mounted directly to the inertial reference 902 and a second axis (in this case, the Y-axis 906) is mounted upon the first axis. As shown in FIGS. 9 and 10, this controls all movement in the horizontal plane that is perpendicular to the page. As shown in FIG. 9, a third axis (in this case, the Z-axis 908) can be mounted upon the second axis and is used to control vertical movement of the target platform 909. The objective 910, in FIG. 9, is coupled to the inertial reference 910 via a structural connection 912 and is thus substantially stationary. In this manner, movement of the target platform 909 relative to the objective 910 is controlled exclusively through movement of the target platform. In an alternative embodiment illustrated in FIG. 10, the Z-axis 908 is instead used to control movement of the objective 910 relative to the target platform 909, and is therefore coupled to the inertial reference 902 via the structural connection 912.

However, both embodiments illustrated in FIGS. 9 and 10 will be limited by the relatively low resonant frequencies that will be included in the structural loop (i.e., the physical connections between the objective 910 and the target platform 909) by virtue of the relatively long and flexible structural connection 912. As a result, performance of the systems illustrated in 9 and 10 will be limited because each movement of the target platform 909 and/or the objective 910 will induce relatively low resonant frequencies that will take proportionately longer to dissipate, thereby limiting the speed and/or accuracy of the system.

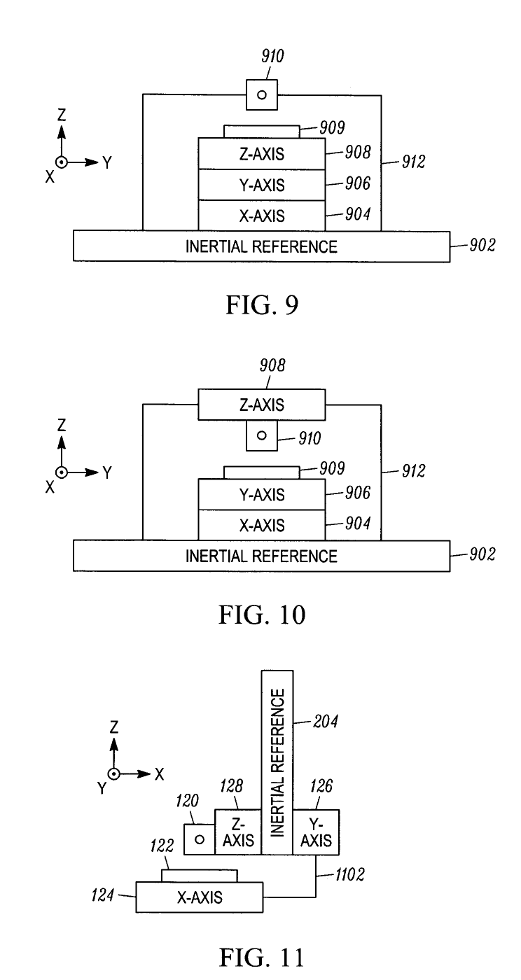

To address such limitations, the structural system illustrated in FIG. 11 may be employed. As shown, two of the axes of control, in this case the Y-axis 126 and the Z-axis 128 are mounted directly to the inertial reference 204, thereby allowing both axes to benefit from tight coupling to the relatively high resonant frequency of the inertial reference 204. To achieve this, it is preferred to mount the inertial reference in a vertical orientation as shown. This is particularly useful in order to reduce the overall footprint of the sequencer 100 and to provide particularly precise control of the objective 120 via the Z-axis 128. Additionally, interposing the inertial reference 204 between the Z-axis 128 and the Y-axis 126 allows the X-axis 124 (and the target platform 122) to be coupled to the Y-axis via a relative short and stiff structural connection 1102, thereby minimizing any relatively low resonant frequencies in the resulting structural loop.

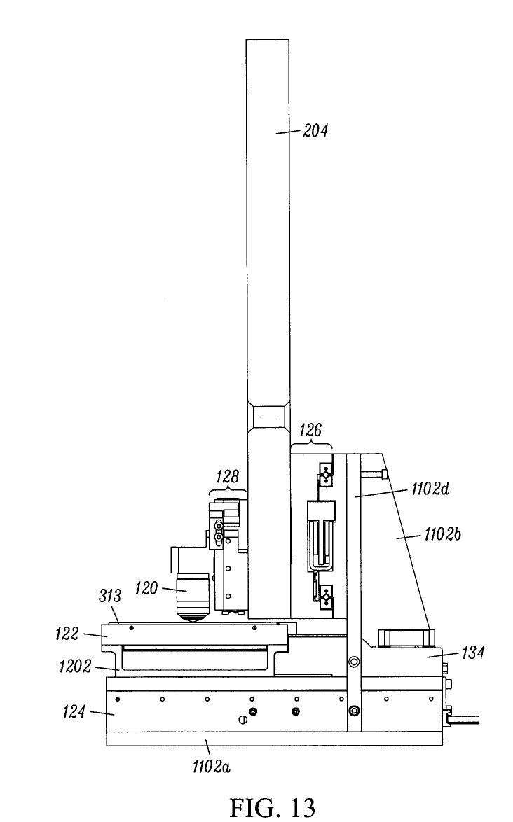

An implementation of the structure shown in FIG. 11 is further illustrated in FIGS. 12 and 13. As shown, the inertial reference 204 is vertically mounted (on the vertical supports 206, not shown) with the Z-axis 128 directly mounted on a front face thereof. As best shown in FIG. 13, the Y-axis 126 is directly mounted on a back face of the inertial reference 204, with the X-axis 124 coupled to the Y-axis 126 by virtue of the structural connection 1102. As shown, the target platform 122 is mounted to the X-axis 124 by a trio of short, stiff supports 1202 that allow the temperature controller 134 to be mounted in proximity to the target platform 122 (and the corresponding flow cells 313). In the illustrated embodiment, the structural connection 1102 between the Y-axis 126 and the X-axis 124 comprises a bottom plate 1102a coupled to side walls 1102b, 1102c and a rear plate 1102d. Note that, in FIG. 13, the right side wall 1102c is removed for ease of illustration. Because the structural connection 1102 is relatively short and stiff, the system is capable of very high performance, including rapid movements and short settling times.

As described above, the sequencer of the present invention overcomes many of the limitations of prior art devices. This is achieved, in part, through the use of a fluid transport subsystem, including a mixer for the preparation of desired reagent mixtures, that replaces relatively expensive autosampler equipment and provides no less flexibility in developing desired chemistry protocols. Furthermore, quick and reliable selection of filter cubes is provided through the use of a highly reliable and precise beamsplitter indexer. Further still, a motion control system is provided that allows for high performance throughput in an apparatus having a relatively small footprint.

While particular preferred embodiments have been shown and described, it will be obvious to those skilled in the art that changes and modifications may be made without departing from the instant teachings. For example, the sequencer described herein may be equipped with a suitable user interface that allows a user to determine status of the sequencer. It is therefore contemplated that any and all modifications, variations or equivalents of the above-described teachings fall within the scope of the basic underlying principles disclosed above and claimed herein.

What is claimed is:

1. In a genome sequencer, a system for transporting fluids comprising:
a mixing chamber;
storage for a plurality of reagents;
a multi-port valve in fluid communication with the storage;
a multi-port pump in fluid communication with the multi-port valve and the mixing chamber, the multi-port pump operable to draw at least one reagent of the plurality of reagents from the storage via the at least one multi-port valve into the mixing chamber; and
a flow cell in fluid communication with the multi-port pump and the mixing chamber, wherein the multi-port pump is operable to draw a mixture comprising the at least one reagent from the mixing chamber into the flow cell.

2. The system for transporting fluids of claim 1, wherein the storage further comprises a plurality of separate storage components, the system further comprising:
at least two multi-port valves, each of the at least two multi-port valves in fluid communication with different portions of the plurality of separate storage components.

3. The system of claim 2, wherein the plurality of storage components comprise a storage block, the at least two multi-port valves further comprising:
a first multi-port valve in fluid communication with the multi-port pump and a first portion of the storage block, wherein the multi-port pump is operable to draw any of that portion of the plurality of reagents in the first portion of the storage block via the first multi-port valve into the mixing chamber; and
a second multi-port valve in fluid communication with the multi-port pump and a second portion of the storage block, wherein the multi-port pump is operable to draw any of that portion of the plurality of reagents in the second portion of the storage block via the second multi-port valve into the mixing chamber.

4. The system of claim 2, wherein the plurality of storage components comprise a plurality of containers, the at least two multi-port valves further comprising:
a third multi-port valve in fluid communication with the flow cell and the plurality of containers, wherein the multi-port pump is operable to draw any of that portion of the plurality of reagents stored in the plurality of containers via the third multi-port valve into the flow cell.

5. The system of claim 4, the third multi-port valve in fluid communication with a third portion of the storage block, wherein the multi-port pump is operable to draw any of that portion of the plurality of reagents in the third portion of the storage block via the third multi-port valve into the flow cell.

6. The system of claim 4, wherein at least one container of the plurality of containers is external to the genome sequencer.

7. The system of claim 4, wherein at least one container of the plurality of containers is internal to the genome sequencer.

8. The system of claim 1, further comprising:
a waste container in fluid communication with the multi-port pump, wherein the multi-port pump is operable to draw fluids from the flow cell into the waste container.

9. The system of claim 8, wherein the multi-port pump is operable to draw fluids from the multi-port valve into the waste container.

10. In a genome sequencer, a system for transporting fluids comprising:
a manifold;
a multi-port valve mounted upon and in fluid communication with a first side of the manifold;
a plurality of syringe tubes mounted upon and in fluid communication with a second side of the manifold, each of the plurality of syringe tubes in fluid communication with a corresponding port of the multi-port valve via the manifold;
a storage block comprising a plurality of recesses for storage of reagents, each of the plurality of recesses aligned with a corresponding one of the plurality of syringe tubes; and
a movable support supporting the manifold and the storage block such that the plurality of syringe tubes can be moved into and out of fluid communication with the plurality of recesses.

11. The system of claim 10, the movable support further comprising:
at least one vertical post; and
at least one bracket, coupled to the manifold and slidably mounted on the at least one vertical post such that the manifold can be moved vertically while maintaining lateral alignment with the storage block.

12. The system of claim 11, the movable support further comprising:
at least one support panel; and
a lever member rotatably mounted at one end thereof to the support panel and movably coupled to the at least one bracket such that rotation of the lever member induces vertical movement of the bracket.

13. In a genome sequencer, a beamsplitter indexer comprising:
a support member comprising an index indicator;
a plurality of beamsplitters coupled to the support member;
a stepper motor directly coupled to the support member;
a sensor, positioned relative to the support member to detect presence of the index indicator; and
a controller, in communication with the sensor and the stepper motor, operative to control the stepper motor at an initial position upon receiving an indication from the sensor of the presence of the index indicator.

14. The beamsplitter indexer of claim 13, wherein an optical axis of one of the plurality of beamsplitters is aligned with an optical axis of an illumination source when the support member is at the initial position.

15. The beamsplitter indexer of claim 13, wherein no optical axis of any of the plurality of beamsplitters is aligned with an optical axis of an illumination source when the support member is at the initial position.

16. The beamsplitter indexer of claim 13, wherein the support member is a rotor having a center and a peripheral edge at a radial distance from the center.

17. The beamsplitter indexer of claim 16, wherein each of the plurality of beamsplitters is mounted in proximity to the peripheral edge of the rotor.

18. The beamsplitter indexer of claim 16, wherein each of the plurality of beamsplitters is mounted such that a first opening of the beamsplitter is perpendicular to the peripheral edge of the rotor.

19. The beamsplitter indexer of claim 18, wherein each of the plurality of beamsplitters comprises a second opening and a third opening both perpendicular to the first opening, the second opening parallel to and at a distance from the third opening, wherein the second opening and the third opening are aligned with a corresponding opening in the rotor.

20. The beamsplitter indexer of claim 19, wherein an optical axis passing through centers of the second opening and the third opening is vertically aligned.

21. The beamsplitter indexer of claim 13, further comprising:
an alignment member coupled to the stepper motor and sensor and maintaining the stepper motor, support member and sensor in fixed alignment; and
an optical element, coupled to the alignment member in fixed alignment with the support member.

22. The beamsplitter of claim 21, wherein the stepper motor is configured to move between a plurality of fixed positions, and wherein each of the plurality of beamsplitters is positioned relative to a corresponding one of the plurality of fixed positions such that, when the stepper motor is at one of the plurality of fixed positions, a beamsplitter of the plurality of beamsplitters is optically aligned with the optical element.

23. In a genome sequencer, a motion control system comprising:
an inertial reference;
an objective;
a target platform;
a first axis of control, directly coupled to the inertial reference and the objective, operable to control motion of the objective along a first axis;
a second axis of control directly coupled to the inertial reference; and
a third axis of control coupled to the second axis of control and the target platform, the second axis of control operable to control motion of the target platform along a second axis perpendicular to the first axis, and the third axis of control operable to control motion of the target platform along a third axis perpendicular to the first axis and the second axis.

24. The motion control system of claim 23, wherein the inertial reference is vertically oriented.

25. The motion control system of claim 23, further comprising:
a structural support coupled to the second axis of control and the third motion control and configured such that the target platform is in proximity to the objective.

26. The motion control system of claim 23, wherein the first axis is a vertical axis and the second and third axes are horizontal axes.
